(19) United States
(12) Patent Application Publication
(10) Pub. No.: US 2024/0299530 A1
(43) Pub. Date: Sep. 12, 2024

(54) METHOD FOR PRODUCING BLOOD PLASMA PRODUCT USEFUL IN THE TREATMENT OF VIRAL INFECTIONS CHARACTERIZED BY AN UNCONTROLLED RELEASE OF PROINFLAMMATORY CYTOKINES

(71) Applicant: Antnor Limited, Toronto (CA)

(72) Inventors: Anthony Galea, Toronto (CA); Irina Brokhman, Vaughan (CA)

(21) Appl. No.: 18/262,068

(22) PCT Filed: Jan. 17, 2022
(86) PCT No.: PCT/CA2022/050059

§ 371 Date: Jul. 19, 2023

(30) Foreign Application Priority Data
Jan. 20, 2021 (CA) 3106197

(51) Int. Cl. A61K 39/215; A61K 39/00; A61K 47/12; A61P 37/04

(52) U.S. Cl. CPC A61K 39/215; A61K 47/12; A61P 37/04; A61K 2039/505

(57) ABSTRACT

A method of producing blood plasma useful in the treatment of a virus causing a viral infection characterized by an uncontrolled release of proinflammatory cytokines in a human affected by the viral infection comprises the following steps: adding a quantity of sodium citrate to a tube; delivering a blood collected from a human donor having antibodies to the virus to the tube; incubating the blood at a temperature of about 37° C. for about 6 hours to about 24 hours; centrifuging the blood to separate the blood into a plasma component and a cellular fraction; and collecting the plasma component. The plasma component can be administered to patient infected with a virus causing a viral infection characterized by an uncontrolled release of proinflammatory cytokines.

### TECHNICAL FIELD

The application is directed generally to medicine, and more particularly to methods and compositions useful, in among other things, the treatment of viral infections characterized by an uncontrolled release of proinflammatory cytokines.

### BACKGROUND

The outbreak of the severe acute respiratory syndrome coronavirus 2 (SARS-COV-2) disease otherwise known as Covid-19 has spread rapidly within China and throughout the world. Complications and death arising from this virus are associated with excessive or/and uncontrolled release of proinflammatory cytokines. This effect is known as “cytokine storm”. It has been reported previously that cytokine storm correlates directly with tissue injury and an unfavorable prognosis of severe viral influenza. Severe cytokine storm is associated with markedly higher levels of pro-inflammatory cytokines including IL-1. It has been shown that pro-inflammatory cytokine IL-1 drives upregulation of MMP-9 (Matrix Metalloproteinase) enzyme expression and its activation in different cell types, including distal airway epithelium. MMP-9 regulates acute lung injury, disrupts airway epithelial barrier function and degrades a broad spectrum of extracellular matrix (ECM) proteins. A recent study shows that MMP-9 deficiency in lung structural cells protected mice from viral-induced mortality. The enzymatic activities of MMPs are strictly controlled by a family of endogenous inhibitors: TIMP-1,2,3,4.

It is known that in general, TIMPs are secreted proteins that have a positive effect on cell growth and survival. Each protein has a distinct role in regulating MMP enzymes. For instance, TIMP1 has been shown to more effectively inhibit MMP3 and MMP9 than TIMP2 whereas TIMP2 inhibits MMP2 more effectively than TIMP1. It has been shown that pro-inflammatory cytokine IL-1 drives upregulation of MMP enzyme expression and activation in different cell types.

There is a need for effective treatments against COVID-19 and other viruses that cause cytokine storm and for therapeutic strategies for treating viral infections characterized by cytokine storm.

### SUMMARY OF THE DISCLOSURE

The present disclosure is directed to a therapeutic intervention directed at MMP9 and IL-1 suppression using natural endogenous TIMPs and the IL-1 inhibitor, IL-1ra, on the regulation of inflammatory processes in patients suffering from viral infections causing cytokine storm including SARS-COV-2 (COVID-19) patients.

The present disclosure is directed to a method involving collecting blood from a donor. The donor may be an individual that has been immunized from a virus, such as COVID-19, influenza virus and others, that causes cytokine storm. Alternatively, the donor may be the same patient suffering from a viral infection causing cytokine storm to whom the method is directed. In such a case, the method is an autologous procedure. It is possible that the donor may also be an individual that has not been immunized from a virus that causes cytokine storm. The collected blood is preferably incubated for from about 6 to about 24 hours, more preferably from 6 to 12 hours at about 37° C. to about 38° C. Plasma from the donor is then obtained from the incubated blood which is enriched by IL-1ra, TIMP1 and TIMP2. The plasma can then be administered to the patient suffering from a viral infection characterized by cytokine storm, such as COVID-19, in order to alleviate symptoms caused by cytokine storm.

Where the donor is an individual that has been immunized from a virus that causes cytokine storm, the blood plasma from the immunized donor is immunized blood plasma. The immunized blood plasma obtained is enriched by IL-1ra, TIMP1, TIMP2, and also contains antibodies to the virus that causes cytokine storm. The immunized blood plasma can then be administered to a patient suffering from a viral infection characterized by cytokine storm, such as COVID-19, in order to alleviate symptoms caused by cytokine storm.

Where the donor is a patient suffering from a virus that causes cytokine storm, the blood plasma obtained is enriched by IL-1ra, TIMP1 and TIMP2, and is then administered to the same patient in order to alleviate symptoms caused by cytokine storm.

According to one aspect, there is provided a method of producing blood plasma useful in the treatment of a virus causing a viral infection characterized by an uncontrolled release of proinflammatory cytokines in a human affected by the viral infection, the method comprising the following steps: adding a quantity of sodium citrate to a tube; delivering blood collected from a human having antibodies to the virus to the tube; incubating the blood at a temperature of about 37° C. for about 6 hours to about 24 hours; centrifuging the blood to separate the blood into an immunized blood plasma component and a cellular fraction; and collecting the immunized blood plasma component.

According to one aspect, there is provided a method of treating a patient infected with a virus causing a viral infection characterized by an uncontrolled release of proinflammatory cytokines, the method comprising the following steps: collecting blood from a human donor having antibodies to the virus; adding a quantity of sodium citrate to a tube; delivering the blood collected from the human donor having antibodies to the virus to the tube; incubating the blood at a temperature of about 37° C. for about 6 hours to about 24 hours; centrifuging the blood to separate the blood into an immunized blood plasma component and a cellular fraction; collecting the immunized blood plasma component; and administering the immunized blood plasma to the patient.

According to another aspect, there is provided a method of producing blood plasma useful in the treatment of a virus causing a viral infection characterized by an uncontrolled release of proinflammatory cytokines in a patient affected by the viral infection, the method comprising the following steps: adding a quantity of sodium citrate to a tube; delivering a blood collected from the patient to the tube; incubating the blood at a temperature of about 37° C. for about 6 hours to about 24 hours; centrifuging the blood to separate the blood into an immunized blood plasma component and a cellular fraction; and collecting the immunized blood plasma component.

According to yet another aspect, there is provided a method of treating a patient infected with a virus causing a viral infection characterized by an uncontrolled release of proinflammatory cytokines, the method comprising the following steps: collecting blood from the patient; adding a quantity of 4% by weight sodium citrate to a tube; delivering the blood collected from the patient to the tube; incubating the blood at a temperature of about 37° C. for about 6 hours to about 24 hours; centrifuging the blood to separate the blood into an immunized blood plasma component and a cellular fraction; collecting the immunized blood plasma component; and administering the immunized blood plasma to the patient.

According to another aspect, there is provided a method of producing blood plasma useful in the treatment of a virus causing a viral infection characterized by an uncontrolled release of proinflammatory cytokines in a human affected by the viral infection, the method comprising the following steps: adding a quantity of sodium citrate to a tube; delivering a blood collected from a human donor to the tube; incubating the blood at a temperature of about 37° C. for about 6 hours to about 24 hours; centrifuging the blood to separate the blood into a plasma component and a cellular fraction; and collecting the plasma component.

According to another aspect, there is provided a method of treating a human patient infected with a virus causing a viral infection characterized by an uncontrolled release of proinflammatory cytokines, the method comprising the following steps: collecting blood from a human donor; adding a quantity of sodium citrate to a tube; delivering the blood collected from the human donor to the tube; incubating the blood at a temperature of about 37° C. for about 6 hours to about 24 hours; centrifuging the blood to separate the blood into a plasma component and a cellular fraction; collecting the plasma component; and administering the plasma to the patient.

According to another aspect, there is provided the use of a composition having enriched levels of IL-1ra,-TIMP1, and TIMP2 for treating patient suffering from a virus that causes a cytokine storm.

According to another aspect, there is provided the use of a composition having enriched levels of IL-1ra,-TIMP1, TIMP2 and a therapeutically effective level of anti-COVID-19 IgG for treating patient suffering from Covid-19.

According to yet another aspect, there is provided a composition comprising from about 1687 pg/ml to about 3,937 pg/ml of IL-1ra, from about 22,794 pg/ml to about 51,781 pg/ml of TIMP1, from about 22,029 pg/ml to about 32,463 pg/ml of TIMP2, from about 171 pg/ml to about 222 pg/ml of IL-1β, from about 478 pg/ml to about 583 pg/ml of TNFα, from about 3,227 pg/ml to about 3,622 pg/ml of MMP9 and from about 378 ng/ml to about 437 ng/ml of anti-COVID-19 IgG.

According to yet another aspect, there is provided a composition comprising from about 1687 pg/ml to about 3,937 pg/ml of IL-1ra, from about 22,794 pg/ml to about 51,781 pg/ml of TIMP1, from about 22,029 pg/ml to about 32,463 pg/ml of TIMP2 and from about 378 ng/ml to about 437 ng/ml of anti-COVID-19 IgG.

According to another aspect, there is provided a use of a composition comprising from about 1687 pg/ml to about 3,937 pg/ml of IL-1ra, from about 22,794 pg/ml to about 51,781 pg/ml of TIMP1, from about 22,029 pg/ml to about 32,463 pg/ml of TIMP2, from about 171 pg/ml to about 222 pg/ml of IL-1β, from about 478 pg/ml to about 583 pg/ml of TNFα, from about 3,227 pg/ml to about 3,622 pg/ml of MMP9 and from about 378 ng/ml to about 437 ng/ml of anti-COVID-19 IgG for treating patient suffering from a virus that causes a cytokine storm.

According to another aspect, there is provided a use of a composition comprising from about 1687 pg/ml to about 3,937 pg/ml of IL-1ra, from about 22,794 pg/ml to about 51,781 pg/ml of TIMP1, from about 22,029 pg/ml to about 32,463 pg/ml of TIMP2, and from about 378 ng/ml to about 437 ng/ml of anti-COVID-19 IgG for treating patient suffering from a virus that causes a cytokine storm.

According to another aspect, there is provided a composition prepared by the methods described herein for treating a patient suffering from a virus that causes a cytokine storm.

### DESCRIPTION OF THE DRAWINGS

FIG. 1 is a plot of IL-1ra concentration in pg/ml versus time showing a comparison of the mean level of IL-1ra in the blood plasma from 12 healthy human donors who recovered from COVID-19 at different time points following incubation of the donors' blood at 37° C.;

FIG. 2a is a plot of TIMP1 concentration in pg/ml versus time showing a comparison of the mean level of TIMP 1 in the blood plasma from the 12 healthy human donors who recovered from COVID-19 at different time points following incubation of the donors' blood at 37° C.;

FIG. 2b is a plot of TIMP2 concentration in pg/ml versus time showing a comparison of the mean level of TIMP 2 in the blood plasma from the 12 healthy human donors who recovered from COVID-19 at different time points following incubation of the donors' blood at 37° C.;

FIG. 2c is a plot of TIMP1 concentration in pg/ml versus time showing a comparison of the mean level of TIMP1 in the blood plasma from 22 healthy human donors who recovered from COVID-19 at different time points following incubation of the donors' blood at 37° C.;

FIG. 2d is a plot of TIMP2 concentration in pg/ml versus time showing a comparison of the mean level of TIMP2 in the blood plasma from the 22 healthy human donors who recovered from COVID-19 at different time points following incubation of the donors' blood at 37° C.;

FIG. 3 is a plot of IL-1β concentration in pg/ml versus time showing a comparison of the mean level of IL-1β in the blood plasma of the 12 healthy human donors who recovered from COVID-19 at different time points following incubation of the donors' blood at 37° C.;

FIG. 4 is a plot of TNFα concentration in pg/ml versus time showing a comparison of the mean level of TNFα in the blood plasma of the 12 healthy human donors who recovered from COVID-19 at different time points following incubation of the donors' blood at 37° C.;

FIG. 5 is a plot of anti-COVID-19 IgG concentration in ng/ml versus time showing a comparison of the mean level anti-COVID-19 IgG in the blood plasma of the 12 healthy human donors who recovered from COVID-19 at different time points following incubation of the donors' blood at 37° C.;

FIG. 6 is a plot of MMP9 concentration in pg/ml versus time showing a comparison of the mean level of MMP9 in the blood plasma of the 12 healthy human donors who recovered from COVID-19 at different time points following incubation of the donors' blood at 37° C.

FIG. 7 is a plot of IL-1ra concentration in pg/ml versus time showing a comparison of the mean level of IL-1ra in the blood plasma from the 22 healthy human donors who recovered from COVID-19 at different time points following incubation of the donors' blood at 37° C.;

FIG. 8 is a plot of MMP9 concentration in pg/ml versus time showing a comparison of the mean level of MMP9 in the blood plasma from the 22 healthy human donors who recovered from COVID-19 at different time points following incubation of the donors' blood at 37° C.;

FIG. 9 is a plot of IL-1β concentration in pg/ml versus time showing a comparison of the mean level of IL-1β in the blood plasma from the 22 healthy human donors who recovered from COVID-19 at different time points following incubation of the donors' blood at 37° C.;

FIG. 10 is a plot of TNFα concentration in pg/ml versus time showing a comparison of the mean level of TNFα in the blood plasma from the 22 healthy human donors who recovered from COVID-19 at different time points following incubation of the donors' blood at 37° C.; and

FIG. 11 is a plot of anti-COVID-19 IgG concentration in pg/ml versus time showing a comparison of the mean level of anti-COVID-19 IgG in the blood plasma from the 22 healthy human donors who recovered from COVID-19 at different time points following incubation of the donors' blood at 37° C.

### DETAILED DESCRIPTION

The present disclosure is directed to a method for creating an acellular blood product containing an anti-inflammatory/anti-catabolic component that is enriched by IL-1ra (IL-1 antagonist), TIMP1 and TIMP2 (endogenous MMPs inhibitors) based on a detailed cytokines analysis. The method comprises a first step of collecting blood from a human donor. The human donor may be an individual who has developed immunity to a virus that causes a cytokine storm in infected individuals. The blood is preferably collected into a glass tube that that contains a quantity of 4% by weight citric acid which may be in the form of 4% by weight sodium citrate. Preferably the ratio of blood to 4% by weight citric acid is about 9:1. The blood is then incubated preferably for about 6 hours to about 24 hours at a temperature of preferably about 37° C. The blood is then centrifuged at about 4000 rpm for 10 minutes to separate the blood into a supernatant component (plasma) and a cellular fraction. Where the plasma is obtained from the blood of a donor who is immunized to a virus such as Covid-19 that causes cytokine storm, the plasma is immunized blood plasma. The supernatant component is then collected, preferably using a clearly labeled syringe. The supernatant component may be frozen at about −70° C. and/or in liquid nitrogen for storage up to one year. Preferably about 100 ml to about 200 ml of the supernatant component is collected, preferably using a clearly labeled syringe into preferably a 200 ml transfusion bag labeled with date and donor name.

The efficacy and safety of the blood product of the present disclosure has been evaluated by an Institutional Review Board (IRB) approved clinical trial that demonstrated a stable therapeutic effect in the treatment of knee osteoarthritis along with a high safety profile. The method for production of plasma from blood donors of the present disclosure provides an immunomodulatory blood product that targets cytokine storm by downregulating IL-1 and MMP-9 pathways and supports the immune system of a patient infected with a virus causing cytokine storm, with a high neutralizing antibody titer from a donor who has recovered from an infection from the virus. Alternatively, the donor may be the patient suffering from virus causing cytokine storm to whom the method of the present disclosure is directed. The donor could also be a human who does not have antibodies to a virus causing cytokine storm.

In vitro data have shown that SARS-COV-2 (COVID-19) immunized patient plasma contains increased levels of anti-catabolic proteins TIMP1 and TIMP2. Molecular cytokine analysis has shown that applying a method for creating an acellular blood product containing an anti-inflammatory/anti-catabolic component leads to IL-1ra enrichment without affecting an anti-COVID-19 IgG concentration in the final blood product.

### EXAMPLES

The blood product of the present disclosure was prepared from 12 healthy volunteer human donors who recovered from COVID-19 according to the following procedure:
Collecting 100 mL to about 400 mL of blood from the subject into a glass tube that is labeled with subject ID; and containing 4% by weight citric acid, in a ratio of 9:1 (blood:4% by weight citric acid).
Incubating the blood (within the same previously labeled tube) at a temperature of 37° C. for 6 h to 24 h.
Centrifuging the blood at 4000 rpm for 10 minutes to separate the blood into a supernatant component (plasma) and a cellular fraction within the same previously labeled tube.
Collecting a sample from each tube to serology analysis.
Collecting 2.5-3 mL of the plasma/immunized plasma component using a clearly labeled syringe into a 200 ml transfusion bag labeled with date and donor name.
The plasma component may optionally be frozen at −70° C. for storage for up to one year.

### Example 1-IL-1ra Levels

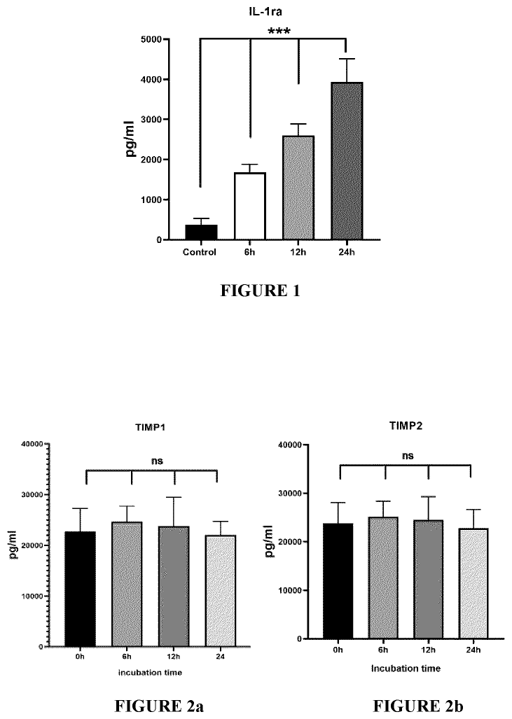

The level of IL-1ra in the plasma obtained from incubated blood samples of 12 recovered COVID-19 patients was measured. The mean IL-1ra levels at different time points as measured in the plasma obtained from the incubated blood of the 12 healthy human donors tested who recovered from COVID-19 are shown in FIG. 1. The results show a significant IL-1ra enrichment starting after 6 hours of incubation at 37° C. as demonstrated by a one-way analysis of variance (ANOVA) test. The results of the measurement of IL-1ra levels at different time points as shown in FIG. 1 are summarized in Table 1.

TABLE 1
Mean IL-1ra Levels Measured at Different
Time Points For 12 Human Donors

| Time | Mean Concentration of IL-1ra |
|---|---|
| (Hours) | (pg/ml) |
| 0 (Control) | 365.934 |
| 6 | 1687.02 |
| 12 | 2,597.95 |
| 24 | 3,936.9 |

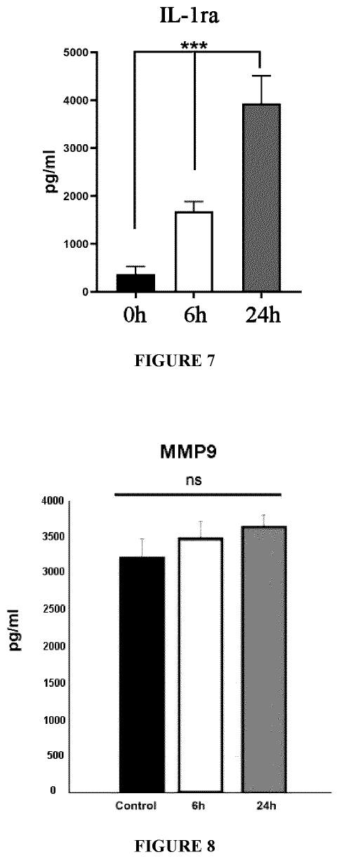

Further data was gathered from 22 healthy human donors who recovered from COVID-19 according to the same procedure. The results of the measurement of IL-1ra levels at different time points for the 22 healthy human donors are shown in FIG. 7 and are summarized in Table 2.

TABLE 2
Mean IL-1ra Levels Measured at Different
Time Points For 22 Human Donors

| Time | Mean Concentration of IL-1ra |
|---|---|
| (Hours) | (pg/ml) |
| 0 (Control) | 286.187 |
| 6 | 1,783.25 |
| 24 | 3,666.96 |

The results from the 22 healthy human donors who recovered from COVID-19 are consistent with the results obtained from the initial 12 healthy human donors tested who recovered from COVID-19.

### Example 2-TIMP1 and TIMP2 Levels

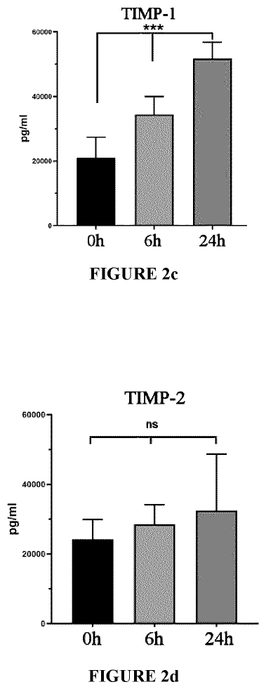

The level of TIMP1 in the plasma obtained from incubated blood samples from recovered COVID-19 patients was measured. The mean TIMP1 levels at different time points as measured in the plasma obtained from the incubated blood of the 12 healthy human donors tested who recovered from COVID-19 are shown in FIG. 2a. Similarly, the level of TIMP2 in incubated blood samples from recovered COVID-19 patients was measured. The mean TIMP2 levels at different time points as measured in the plasma obtained from the incubated blood of the 12 healthy human donors tested who recovered from COVID-19 are shown in FIG. 2b. The results demonstrate that the plasma of healthy donors who recovered from COVID-19 contains increased base line concentrations of TIMP1 and TIMP2 that are not affected by the protocol for producing enhanced anti-inflammatory/catabolic agents from human blood. The average base line TIMP1 concentration in non-exposed COVID-19 individuals is 6,000 pg/ml and the average base line TIMP2 concentration in non-exposed COVID-19 individuals is 3,900 pg/ml, as demonstrated by a one-way analysis of variance (ANOVA) test.

The mean TIMP1 levels at different time points as measured in the plasma obtained from the incubated blood of the 12 healthy human donors tested who recovered from COVID-19 as shown in FIG. 2a are summarized in Table 3.

TABLE 3
Mean TIMP1 Levels Measured at Different
Time Points For 12 Human Donors

| Time | Mean Concentration of TIMP1 |
|---|---|
| (Hours) | (pg/ml) |
| 0 (Control) | 22,742.505 |
| 6 | 24,669.56149 |
| 12 | 23,800.34754 |
| 24 | 22,793.8982 |

The mean TIMP2 levels at different time points as measured in the plasma obtained from the incubated blood of the 12 healthy human donors tested who recovered from COVID-19 as shown in FIG. 2b are summarized in Table 4.

TABLE 4
Mean TIMP2 Levels Measured at Different
Time Points For 12 Human Donors

| Time | Mean Concentration of TIMP2 |
|---|---|
| (Hours) | (pg/ml) |
| 0 (Control) | 23,833.88001 |
| 6 | 25,141.12136 |
| 12 | 24,503.00124 |
| 24 | 22,028.90211 |

Further data was gathered from the 22 healthy human donors who recovered from COVID-19 according to the same procedure. The data from those donors showed a statistically significant increase in the concentration of TIMP1 in the plasma obtained from the incubated blood at 6 hours and 24 hours of incubation of the blood at 37° C. Although the data from the 12 donors related to the TIMP1 measurements shown in FIG. 2a did not show a statistically significant difference at 6 hours and 24 hours of incubation of the blood at 37° C., it is not unusual that a larger sample size of donors yielded different results in this regard. The mean TIMP1 levels at different time points as measured in the plasma obtained from the incubated blood of the 22 healthy human donors tested who recovered from COVID-19 as shown in FIG. 2c are summarized in Table 5

TABLE 5
Mean TIMP1 Levels of 22 Donors Measured
at Different Time Points

| Time | Mean Concentration of TIMP1 |
|---|---|
| (Hours) | (pg/ml) |
| 0 (Control) | 20,962.3 |
| 6 | 34,414.9 |
| 24 | 51,781 |

Further data was gathered from the 22 healthy human donors who recovered from COVID-19 according to the same procedure. The results of the measurement of TIMP2 at different time points for the 22 healthy human donors are shown in FIG. 2d and are summarized in Table 6.

TABLE 6
Mean TIMP2 Levels Measured at Different
Time Points For 22 Human Donors

| Time | Mean Concentration of TIMP2 |
|---|---|
| (Hours) | (pg/ml) |
| 0 (Control) | 24,204.1 |
| 6 | 28,492.6 |
| 24 | 32,462.6 |

The results from the 22 healthy human donors who recovered from COVID-19 are consistent with the results obtained from the initial 12 healthy human donors tested who recovered from COVID-19 in that there is not a statistically significant increase in mean TIMP2 levels.

### Example 3-IL-1β Levels

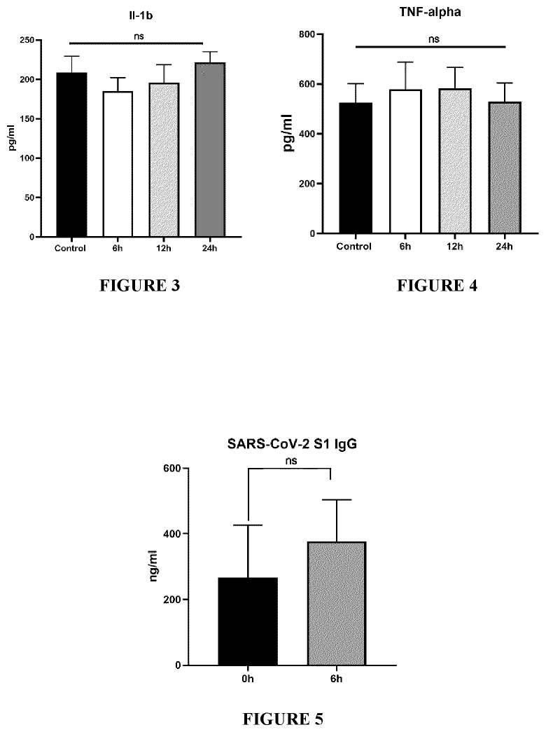

The mean IL-1β levels at different time points as measured in the blood product produced from the 12 healthy human donors tested who recovered from COVID-19 is shown in FIG. 3. The results show that the procedure does not cause an upregulation of pro-inflammatory cytokine IL-1β concentrations in the healthy donors who recovered from COVID-19, as demonstrated by a one-way analysis of variance (ANOVA) test. The results of the measurement of IL-1β levels at different time points are shown in FIG. 3 and are summarized in Table 7.

TABLE 7
Mean IL-1β Levels Measured at
Different Time Points For 12 Human Donors

| Time | Mean Concentration of IL-1β |
|---|---|
| (Hours) | (pg/ml) |
| 0 (Control) | 208.378 |
| 6 | 184.905 |
| 12 | 195.708 |
| 24 | 221.752 |

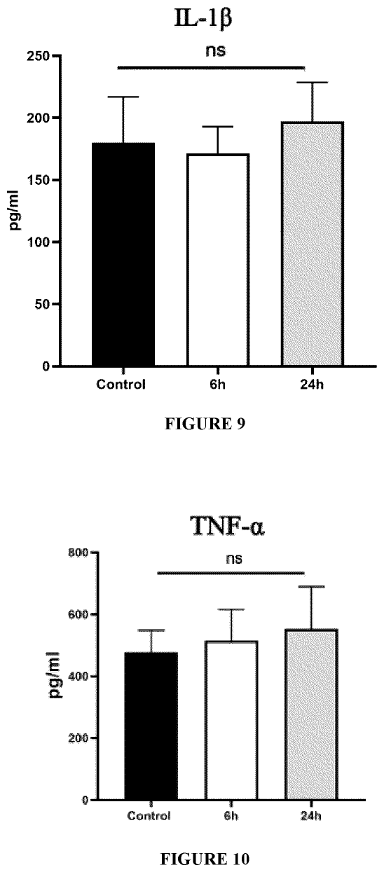

Further data was gathered from the 22 healthy human donors who recovered from COVID-19 according to the same procedure. The results of the measurement of IL-1β levels at different time points for the 22 healthy human donors are shown in FIG. 9 and are summarized in Table 8.

TABLE 8
Mean IL-1β Levels Measured at
Different Time Points For 22 Human Donors

| Time | Mean Concentration of IL-1β |
|---|---|
| (Hours) | (pg/ml) |
| 0 (Control) | 180.076 |
| 6 | 171.322 |
| 24 | 197.404 |

The results from the 22 healthy human donors who recovered from COVID-19 are consistent with the results obtained from the initial 12 healthy human donors tested who recovered from COVID-19.

### Example 4-TNFα Levels

The mean TNFα levels at different time points as measured in the blood product produced from the 12 healthy human donors tested who recovered from COVID-19 is shown in FIG. 4. The results show that the procedure does not cause an upregulation of pro-inflammatory cytokine TNFα concentrations in the healthy donors who recovered from COVID-19, as demonstrated by a one-way analysis of variance (ANOVA) test. The results of the measurement of TNFα levels at different time points shown in FIG. 4 are summarized in Table 9.

TABLE 9
Mean TNFα Levels Measured at Different
Time Points For 12 Human Donors

| Time | Mean Concentration of TNFα |
|---|---|
| (Hours) | (pg/ml) |
| 0 (Control) | 525.034 |
| 6 | 579.295 |
| 12 | 582.749 |
| 24 | 529.142 |

Further data was gathered from the 22 healthy human donors who recovered from COVID-19 according to the same procedure. The results of the measurement of TNFα levels at different time points for the 22 healthy human donors are shown in FIG. 10 and are summarized in Table 10.

TABLE 10
Mean TNFα Levels Measured at Different
Time Points For 22 Human Donors

| Time | Mean Concentration of TNFα |
|---|---|
| (Hours) | (pg/ml) |
| 0 (Control) | 477.759 |
| 6 | 515.274 |
| 24 | 552.886 |

The results from the 22 healthy human donors who recovered from COVID-19 are consistent with the results obtained from the initial 12 healthy human donors tested who recovered from COVID-19.

### Example 5-anti-COVID-19 IgG Levels

The mean levels of anti-COVID-19 IgG at 0 hours and 6 hours as measured in the blood product produced from the 12 healthy human donors tested who recovered from COVID-19 is shown in FIG. 5. The results show that the procedure for producing the blood product from donor patients who recovered from SARS-COV-2 (COVID-19) does not downregulate anti-COVID-19 IgG concentration. The results of the measurement of anti-COVID-19 IgG levels at different time points shown in FIG. 5 are summarized in Table 11.

TABLE 11
Mean anti-COVID-19 IgG Levels Measured at
Different Time Points For 12 Human Donors

| Time | Mean Concentration of anti-COVID-19 |
|---|---|
| (Hours) | IgG (ng/ml) |
| 0 (Control) | 266.262 |
| 6 | 377.563 |

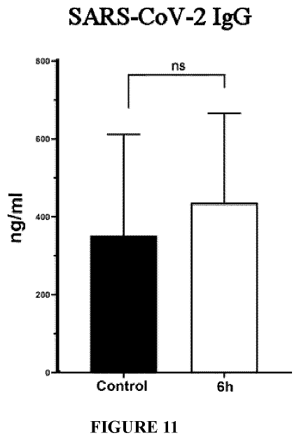

Further data was gathered from the 22 healthy human donors who recovered from COVID-19 according to the same procedure. The results of the measurement of anti-COVID-19 IgG levels at different time points for the 22 healthy human donors are shown in FIG. 11 and are summarized in Table 12.

TABLE 12
Mean anti-COVID-19 IgG Levels Measured at
Different Time Points For 22 Human Donors

| Time | Mean Concentration of anti-COVID-19 |
|---|---|
| (Hours) | IgG (ng/ml) |
| 0 (Control) | 351.922 |
| 6 | 436.961 |

The results from the 22 healthy human donors who recovered from COVID-19 are consistent with the results obtained from the initial 12 healthy human donors tested who recovered from COVID-19.

### Example 6-MMP9 Levels

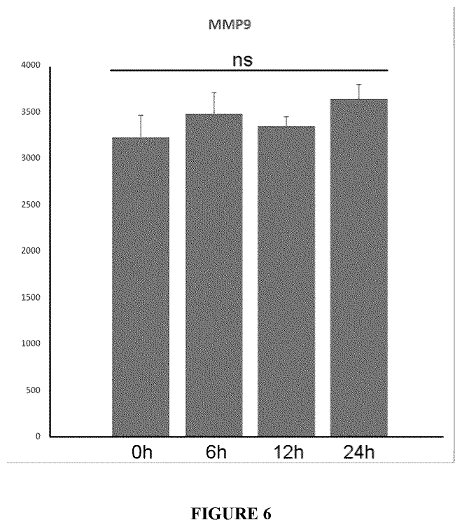

The levels of MMP9 in the plasma obtained from incubated blood samples of the 12 recovered COVID-19 patients were measured. The mean MMP9 levels at different time points as measured in the plasma obtained from the incubated blood of the 12 healthy human donors tested who recovered from COVID-19 are shown in FIG. 6. The results show that the procedure does not cause an upregulation of pro-inflammatory cytokine MMP9 concentrations in the healthy donors who recovered from COVID-19, as demonstrated by a one-way analysis of variance (ANOVA) test. The results of the measurement of MMP9 levels at different time points shown in FIG. 6 are summarized in Table 13.

TABLE 13
Mean MMP9 Levels Measured at Different
Time Points For 12 Human Donors

| Time | Concentration of MMP9 |
|---|---|
| (Hours) | (pg/ml) |
| 0 (Control) | 3,280.34 |
| 6 | 3,449.87 |
| 12 | 3,310.67 |
| 24 | 3,506.69 |

Further data was gathered from the 22 healthy human donors who recovered from COVID-19 according to the same procedure. The results of the measurement of MMP9 levels at different time points for the 22 healthy human donors are shown in FIG. 8 and are summarized in Table 14.

TABLE 14
Mean MMP9 Levels Measured at Different
Time Points For 22 Human Donors

| Time | Mean Concentration of MMP9 |
|---|---|
| (Hours) | (pg/ml) |
| 0 (Control) | 3,226.71 |
| 6 | 3,448.65 |
| 24 | 3,622.36 |

The results from the 22 healthy human donors who recovered from COVID-19 are consistent with the results obtained from the initial 12 healthy human donors tested who recovered from COVID-19.

### CONCLUSIONS

Incubated blood of the donor patients who recovered from COVID-19 is a source of anti-inflammatory/catabolic and regenerative agents. COVID-19 (SARS-COV-2) immunized patient plasma contains increased levels of anti-catabolic proteins TIMP1, TIMP2 and anti-inflammatory IL-1ra after 6 hours of incubation. Applying the method for creating an acellular blood product containing an anti-inflammatory/anti-catabolic component leads to IL-1ra enrichment with no upregulation of pro-inflammatory TNFα, Il-1B and MMP9. Applying the method for creating acellular blood product containing an anti-inflammatory/anti-catabolic component does not downregulate anti-COVID-19 IgG concentration.

In vitro results from the initial 12 donors and the further 22 donors demonstrate that the production of immunized plasma from COVID-19 immunized patients is effective in the treatment of COVID-19 induced cytokine storm through the administration of the immunized plasma to a patient suffering from COVID-19 or another viral infection that causes a cytokine storm.

Although the invention has been described with reference to illustrative embodiments, it is to be understood that the invention is not limited to these precise embodiments. Numerous modifications, variations, and adaptations may be made to the particular embodiments of the invention described above without departing from the scope of the invention. The scope of the claims should not be limited by the preferred embodiments set forth in the examples, but should be given the broadest interpretation consistent with the description as a whole.

1-5. (canceled)

6. A method of treating a patient infected with a virus causing a viral infection characterized by an uncontrolled release of proinflammatory cytokines, the method comprising the following steps:
collecting blood from a human donor having antibodies to the virus;
adding a quantity of sodium citrate to a tube;
delivering the blood collected from the human donor having antibodies to the virus to the tube;
incubating the blood at a temperature of about 37° C. for about 6 hours to about 24 hours;
centrifuging the blood to separate the blood into an immunized blood plasma component and a cellular fraction;
collecting the immunized blood plasma component; and
administering the immunized blood plasma to the patient.

7. The method according to claim 6 wherein the blood is incubated for about 6 hours to about 12 hours.

8. The method according to claim 6 wherein the blood is incubated for about 6 hours.

9. The method according to claim 6 wherein the sodium citrate is 4% by weight sodium citrate.

10. The method according to claim 9 wherein the blood collected is delivered to the tube in a ratio of 9:1 blood to the 4% by weight sodium citrate.

11-15. (canceled)

16. A method of treating a patient infected with a virus causing a viral infection characterized by an uncontrolled release of proinflammatory cytokines, the method comprising the following steps:
collecting blood from the patient;
adding a quantity of 4% by weight sodium citrate to a tube;
delivering the blood collected from the patient to the tube;
incubating the blood at a temperature of about 37° C. for about 6 hours to about 24 hours;
centrifuging the blood to separate the blood into an immunized blood plasma component and a cellular fraction;
collecting the immunized blood plasma component; and
administering the immunized blood plasma to the patient.

17. A composition comprising from about 1687 pg/ml to about 3,937 pg/ml of IL-1ra, from about 22,794 pg/ml to about 51,781 pg/ml of TIMP1, from about 22,029 pg/ml to about 32,463 pg/ml of TIMP2, from about 171 pg/ml to about 222 pg/ml of IL-1β, from about 478 pg/ml to about 583 pg/ml of TNFα, from about 3,227 pg/ml to about 3,622 pg/ml of MMP9 and from about 378 ng/ml to about 437 ng/ml of anti-COVID-19 IgG.

18. Use of a composition according to claim 17 for treating patient suffering from a virus that causes a cytokine storm.

19. Use according to claim 18 wherein the virus is COVID-19.

20-24. (canceled)

25. The method according to claim 16 wherein the blood is incubated for about 6 hours to about 12 hours.

26. The method according to claim 25 wherein the blood is incubated for about 6 hours.

27. The method according to claim 25 wherein the sodium citrate is 4% by weight sodium citrate.

28. The method according to claim 27 wherein the blood collected is delivered to the tube in a ratio of 9:1 blood to the 4% by weight sodium citrate.
